(19) United States
(12) Patent Application Publication
(10) Pub. No.: US 2026/0232931 A1
(43) Pub. Date: Aug. 13, 2026

(54) SPACER VALVE AND SYSTEM AND METHOD FOR OPTIMIZATION SPACER VALVE

(71) Applicants: Shahab Azimi, Coquitlam (CA); Siamak Arzanpour, North Vancouver (CA)

(72) Inventors: Shahab Azimi, Coquitlam (CA); Siamak Arzanpour, North Vancouver (CA)

(21) Appl. No.: 19/151,598

(22) PCT Filed: Jan. 21, 2024
(86) PCT No.: PCT/CA2024/050066

§ 371 Date: Jul. 28, 2025

Related U.S. Application Data
(60) Provisional application No. 63/483,300, filed on Feb. 5, 2023.

(51) Int. Cl. A61M 15/00; A61M 11/00; A61M 16/00; A61M 16/20

(52) U.S. Cl. CPC A61M 15/0016; A61M 11/003; A61M 15/0086; A61M 15/009; A61M 16/208; A61M 2016/003; A61M 2206/14; A61M 2230/40

(57) ABSTRACT

Examples of spacer's valve optimized to filter large particles are described. The valve comprises an inlet mounted to an outlet of a spacer body, an outlet configured as a mouthpiece to be positioned into user's mouth, a body that defines an inner cavity extending between the inlet and the outlet and an impact filter positioned in the inner cavity of the valve body. The impact filter has an impact body that has a size/diameter that is larger than a size of the inlet and smaller than a size of the valve inner cavity thus creating a gap/space through which a medication flow stream of particles passes to the outlet. The impact filter is positioned at a distance from the inlet such that a majority of medication's large particles are filtered out of medication flow stream by impacting the impact body while the medication small particles pass through the space/gap into the outlet.

### FIELD OF INVENTION

This invention relates to a spacer's valve optimized to filter large particles and a spacer's valve optimized and adjustable for different inhalation flow rates.

### BACKGROUND OF INVENTION

Unless otherwise indicated herein, the materials described in this section are not prior art to the claims in this application and are not admitted to be prior art by inclusion in this section.

Inhalation therapy in Asthma and Chronic Obstructive Pulmonary Disease (COPD) has several advantages over systemic therapy (i.e., treatment using substances that travel through the bloodstream, reaching and affecting cells all over the body), including but not limited to efficient medication delivery, rapid treatment, and reduced adverse effects due to directing the medication to the lung. Pressurized meter dose inhalers (pMDIs) have been the preferred treatment for respiratory diseases such as asthma and Chronic Obstructive Pulmonary Disease (COPD). Generally, number of studies have been shown that the particles' size and dosage directly influence the effectiveness of inhalation therapy, indicating that particle sizes between 2 and 6 μm are the only portion of the medication absorbed in the lungs. Larger particles would likely deposit in the mouth and esophageal regions and can be eventually swallowed. Valve holding chambers/spacers are accessory devices to pMDIs to capture larger medication particles and prevent their deposition in the oral cavity and upper airways. However, current spacers' effectiveness in capturing large particles is limited. In addition, some studies have shown that around 32 to 96% of users do not use their pMDI according to the manufacturers' instructions, such as for example, synchronization of inhalation and pMDI actuation which are not often followed by the users.

Apart from lower efficiency due to misuse, pMDI has an inherent drawback related to its high-velocity aerosol emission, which causes the impaction of even the fine particles to the upper airways. Typically, the spacers address pMDI issues by increasing the distance between the medication container (e.g., inhaler canister) and the user's mouth and reducing the velocity of aerosols before entering the respiratory system. They are tube-shaped add-on devices equipped with a valve (mouthpiece) at the mouth. Most spacers are also equipped with valves (also called valved-holding chambers) to hold the medication inside the spacer body and give users enough time for better synchronization and multiple inhalations in each pMDI actuation. This feature is highly advantageous for kids and the elderly with low tidal volume. Despite the advantages of valved-holding chambers, a considerable amount of the large particles medication still leaves the spacers and reaches the upper airways.

In addition, breathing flow rate is time-dependent and varies for different people with distinct conditions. Human respiratory system exhibits a wide range of variances in inhalation speed, tidal volume, and lung peak flow rate. Peak flow rate is a typical quantity that may describe individual differences in lung suction capabilities. Age, gender, and height are some frequent factors in determining a healthy person's Peak Expiratory Flow (PEF). PEF in adults aged 15 to 85 varies between 300 and 670 L/min and in infants and children the range is 87 to 393 L/min. FIG. 1 illustrate normal value for PEF for healthy men and women depending on age and height. People suffering with asthma and COPD have a lower peak flow rate than a healthy individual. Basic principles in fluid dynamics suggest that the flow rate is the most critical factor in setting the fluid regime and the behavior of suspended particles.

The spacers and valves known in the prior art can trap fine particles lowering the effectiveness of the therapy while still allow many large particles at the spacer's outlet thus causing deposition of such harmful large particles in the mouth cavity that can be eventually swallowed by the user. In addition, there is no guarantee for such known spacers to still perform optimally in a relatively wide range of flow rates in human inhalation. More specifically, it must be expected that different inhalation flow rates affect the amount of medicine deposited on spacers, the oral cavity, and hence the portion of medicine particles that finally reaches to the intended locations inside the lungs.

### SUMMARY OF THE INVENTION

In one aspect, an impact valve for a spacer is provided. The valve comprises an inlet nozzle mounted to an outlet of a spacer body, an outlet configured as a mouthpiece to be positioned into user's mouth, an elongated tubular body with an inner cavity that extends between the inlet and the outlet and an impact filter that is positioned and secured in the inner cavity of the valve body. The impact filter has a diameter larger than a diameter of the inlet nozzle and smaller than a diameter of the outlet. During inhalation, a majority of medication large particles are filtered out of a medication flow stream by impacting the impact filter while medication small particles pass to the outlet.

In one aspect, the valve comprises a first spring with a predefined spring stiffness mounted into the inner cavity of the valve body. The impact filter is secured to a first end of the spring facing the inlet nozzle. A second spring is also provided with its first end engaging a second end of the first spring. The second spring has a predefined stiffness that is greater than the stiffness of the first spring. During inhalation the first spring is compressed due to lower stiffness moving the impact filter away from the inlet nozzle while the second spring is minimally compressed due to its greater stiffness therefore minimizing a drag force experienced in the valve.

In another aspect, pressurized meter dose inhaler is provided. The inhaler comprises an inhaler container comprising a pressurized aerosolized medication and has an exit port to discharge the aerosolized medication and a spacer that is coupled to the exit port at its first end. The spacer has a body that extends between the spacer first end and its second end. An impact valve is also provided that comprises an inlet nozzle mounted to the second end of the spacer, an outlet configured as a mouthpiece to be positioned into user's mouth, an elongated tubular body that defines an inner cavity which extends between the inlet and the outlet and an impact filter that is positioned in the inner cavity of the valve body, The impact filter has a diameter that is larger than a diameter of the inlet nozzle and smaller than a diameter of the outlet. During inhalation, a majority of medication large particles are filtered out of a medication flow stream by impacting the impact filter while medication small particles pass to the outlet.

In yet another aspect, a peak flow meter is provided. The peak flow meter comprises a valve that comprises an inlet nozzle, an outlet configured as a mouthpiece and an elongated tubular body that defines an inner cavity that extends between the inlet and the outlet. A first spring with a predefined spring stiffness is mounted into the inner cavity of the valve body. The spring has a first end that is facing the inlet nozzle and a second end. A second spring with a first end that engages the second end of the first spring is also provided. The second spring has a predefined stiffness that is greater than the stiffness of the first spring. An impact body is mounted to the first end of the first spring facing the inlet nozzle. The impact body has a diameter larger than a diameter of the inlet nozzle and smaller than a diameter of the outlet. The peak flow meter further comprises a scale coupled to an outer wall of the valve body. The scale is calibrated to measure air mass flow rate and quantify expiratory peak flow. During measurements, an exhalation peak flow compresses the first spring to a greater compression rate due to its lower stiffness while the second spring is engaged and minimally compressed due to its greater stiffness compensating for a force of the exhalation peak flow. A peak flow rate is measured by measuring a distance of the impact body from the valve inlet nozzle.

In addition to the aspects and embodiments described above, further aspects and embodiments will become apparent by reference to the drawings and study of the following detailed description.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the disclosure. Sizes and relative positions of elements in the drawings are not necessarily drawn to scale. For example, the shapes of various elements and angles are not drawn to scale, and some of these elements are arbitrarily enlarged and positioned to improve drawing legibility.

FIG. 1 is a graph of normal value for peak expiratory flow (PEF) for men and women depending on age and height.

FIG. 2 is an example of an impact valve according to an embodiment of the present invention.

FIG. 3 is an schematic view illustrating an airflow stream of large particles trajectory and small particles trajectory based on an impact theory.

FIG. 4 shows a simulation of a valve geometry with a portion of a spacer body.

FIG. 5 shows a simulation of a valve visualizing impact filter efficiency to filter out large particles from an airflow inhaled from the spacer.

FIG. 6 illustrates simulated results of impact valve efficiency to filter large particles comparing to known spacer's valves.

FIG. 7 illustrates a graph of valve's efficiency at various mass flow rates and different filter plate distance.

FIG. 8 shows a graph of relationship between inhalation mass flow rate and filter plate distance to inlet nozzle for flow rates between 10 to 35 L/min.

FIG. 9 is a schematic view of an example of a valve according to another embodiment of the present invention adjustable for different inhalation flow rates.

FIG. 10 is a graph illustrating a drag force on the filter impact plate at different filter plate distance to inlet nozzle for a range of the inhalation mass flow rate.

FIG. 11 is a graph illustrating an inhalation mass flow rate relative to a drag force that is equal to a spring force that carries an impact filter.

FIG. 12 is a graph illustrating results of the two optimal spacer designs (Design 1 and Design 2) in comparison to six commercial spacer designs.

FIG. 13 shows examples of four optimized spacer's body designs.

FIG. 14 is a schematic partial view of a spacer with a valve with an impact filter used as a peak flow meter.

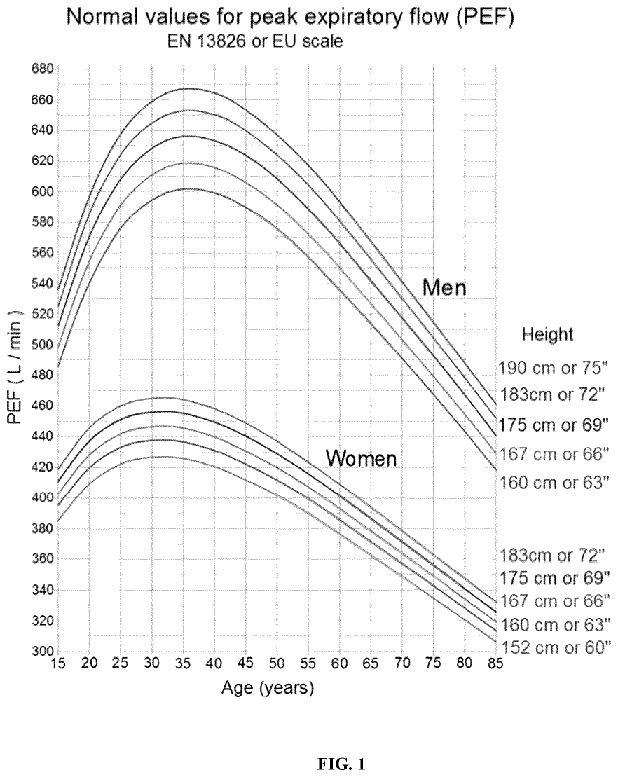

### DETAILED DESCRIPTION OF SPECIFIC EMBODIMENTS

The present invention describes valve for pressurized meter dose inhalers (pMDIs) optimized to filter large particles while passing fine particles. In addition, the present invention discloses a valve for pMDI that is adjustable for different inhalation flow rates. The valve of the present invention, filters larger-sized medication particles that are harmful to humans if ingested, while increases the fine particle medicine particles inhaled by the user. The valve design of the present invention is based on the inertial impaction theory and principle that airflow forces particles with small inertia (fine particles) to follow the airflow streamlines of large particles and causes large and heavier particles with high inertia to be absorbed by impacting a bluff body.

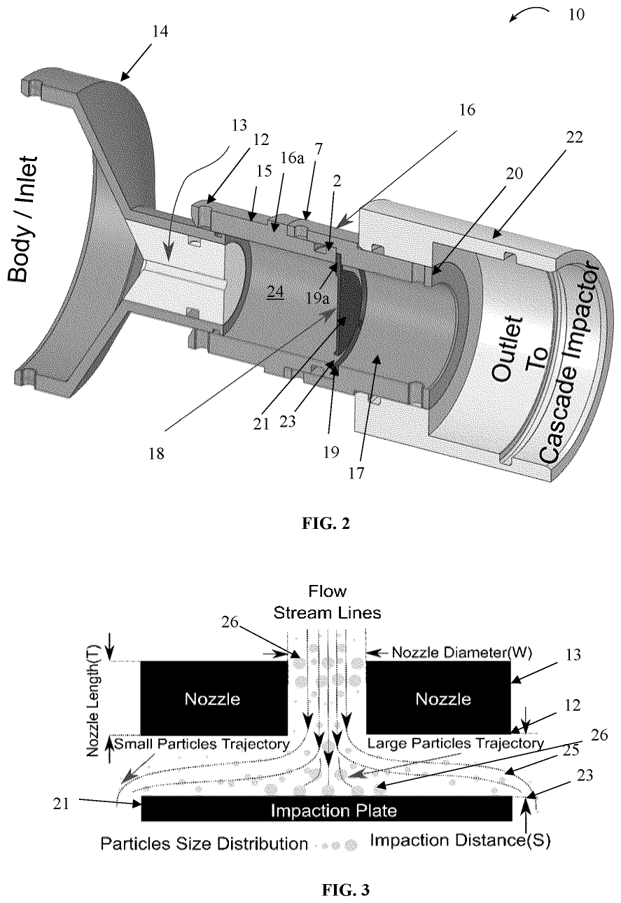

FIG. 2 illustrates an example of a valve 10 according to an embodiment of the present invention. For example, the valve 10 can be an one-way valve. The valve 10 comprises a valve body 16, an inlet 12 formed at a first end of the valve body 16 and an outlet 20 formed a second end of the valve body 16. The valve 10 can be designed as a spacer valve. FIG. 2 illustrates that the inlet 12 of the valve 10 can be coupled to a spacer 14 and can comprise an inlet nozzle 13 that directs the flow of particles form the spacer 14 into the valve body 16. The nozzle 13 is formed in the inlet 12 of the valve 10 and can be integrated as part of the spacer outlet or into the valve inlet 12. In some embodiments, the spacer 14 can be omitted and the valve 10 can be coupled to the nozzle of a pressurized canister of the pMDI device that contains a mixture of an active medication and a propellant. A mouthpiece 22 can be mounted to the outlet 20. The valve body 16 can be elongated tube, such as for example cylindrically shaped tube having a wall 16a defining an inner cavity 24. An impact filter 18 can be mounted into the inner cavity 24. The impact filter 18 can comprise a fastener 19 to secure the impact filter to inner side of the wall 16a of the body 16 and an impact body/plate 21. The fastener 19 can be ring-shaped fastener secured to the body's wall 16a and can further comprise at least one link 19a to connect to the impact body 21. The impact body 21 can have shape conforming/similar to the cross-sectional shape of the inner cavity but smaller in size. For example, if the cross-section shape of the inner cavity is circular, the impact body 21 can be a circular plate with a diameter smaller than the diameter of the inner cavity 24 of the valve body 16. The fastener 19 is spaced apart and surrounding the plate 21 such that a space/gap 23 is formed between the circumferential edge of the impact plate 21 and the fastener 19. Two or more links 19a, separated one from another, can be provided to secure the impact plate 21 to the fastener 19. The size of the impact body/plate 21 of the filter 18 is smaller than the size of the inner cavity 24 in order to form the space/gap 23 for the smaller medication particle to pass the filter 18 toward the outlet 20. The diameter of the impact body 21 should be smaller than the diameter of the outlet 20 but larger than the diameter of the inlet nozzle 13. The diameter of the outlet 20 should be small enough to go inside the user's mouth or to be inserted into the mouthpiece 22.

The body 16 can be configured such that a length of the body 16 can be adjusted to smaller or larger lengths. For example, the valve body 16 can comprise a number of nesting parts that slide one in relation to another to adjust a length of the body 16 and therefore distance of the impact filter 18 to the inlet 12. The body 14 can comprise a first part 15 and a second part 17 slidingly joined together to form the body 16. The first and the second parts 15, 17 can slide one in relation to another to adjust the length of the body 16 and thus adjust the distance of the impact filter 18 from the inlet 12. The first part 15 has a first end at the inlet 12 and a second distal end 2. The second part 17 has a first part 7 and a second end at the outlet 20. The impact filter 18 can be fastened to the second part 17 at a predetermined position between the first end 7 and the outlet 20. The first and the second parts 15, 17 can be slidably coupled such that the second distal end 2 of the first part 15 can slide into the cavity of the second part and reduce the distance between the impact filter 18 and the inlet 12 and the nozzle 13. For example, FIG. 2 shows an example of the valve 10 when the first and the second parts 15, 17 of the valve body are in the nested position and the impact filter is in a closest position to the nozzle 13. As illustrated in FIG. 2, when in the nested position, the end face of the second end 2 of the first part 15 can be adjacent to the fastener 19 of the impact filter 18. When the first and the second parts 15, 17 are in extended position, the impact filter 18 is in its most distant position from the inlet 12 of the valve 10 and the second end 2 of the first part 15 is away from the impact filter 18 and in proximity to the first end 7 of the second part 17. The position of the first and the second parts 15, 17 can be locked one with respect to another. For example, a flange can be formed at the end 2 of the first part 15 that can fit into a groove formed on the inner side of the second part 17 to lock the positioned of the parts 15 and 17. In another example, a number of holes can be formed along a length of the first and second parts 15, 17 of the valve body 16. The first part 15 can slide into part 17 until one of the holes formed in the first part 15 aligns with a hole formed in the wall of the second part and then their position can be locked using a locking pin that is inserted through the aligned opening. In yet another example, the body of the first and the second parts 15, 17 of the valve body 16 can be threaded and the two parts can be connected by screwing them together.

FIG. 3 illustrates airflow stream of medication coming out from the inlet 12 through the nozzle 13 showing that particles with small inertia (e.g., fine particles 25) follow the airflow streamlines of large particles 26 such that the larger particles 26 impact and stick to the impact body 21 while small particles 25 pass through the space 23 in the impact filter 18. The efficiency of the impact filter 18 can be impacted by factors such as for example, relationship between flow dynamics and impaction geometry which depends on the nozzle dimension and shape, distance of the impact filer 18 from the nozzle, particle density, airflow velocity.

Number of experiments were conducted varying nozzle length, nozzle diameter, impact plate distance to valve inlet nozzle, plate diameter, and valve outlet diameter are defined as valve design variables. Valve prototype was designed and manufactured with an outlet diameter of 20 mm, exchangeable inlet nozzle with 2, 5, 8, and 16 mm diameter; and 8, 12, and 16 mm diameter of the impaction plates with continuously adjustable distance from 0 to 30 mm. Four combinations of valve parameters are chosen to cover a meaningful range of design variables. Table. 1 summarizes the design parameters of these valve combinations.

TABLE 1
design parameters on four different valve
combinations for test experiments 1 to 4

| | Nozzle | Nozzle | Plate | Plate | Outlet |
|---|---|---|---|---|---|
| Parameter(mm) | Diameter | Length | Distance | Diameter | Diameter |
| Comb. 1 | 16 | 20.45 | 5 | 16 | 20 |
| Comb. 2 | 8 | 18.14 | 5 | 12 | 20 |
| Comb. 3 | 5 | 17.27 | 5 | 12 | 20 |
| Comb. 4 | 2 | 16.41 | 5 | 8 | 20 |

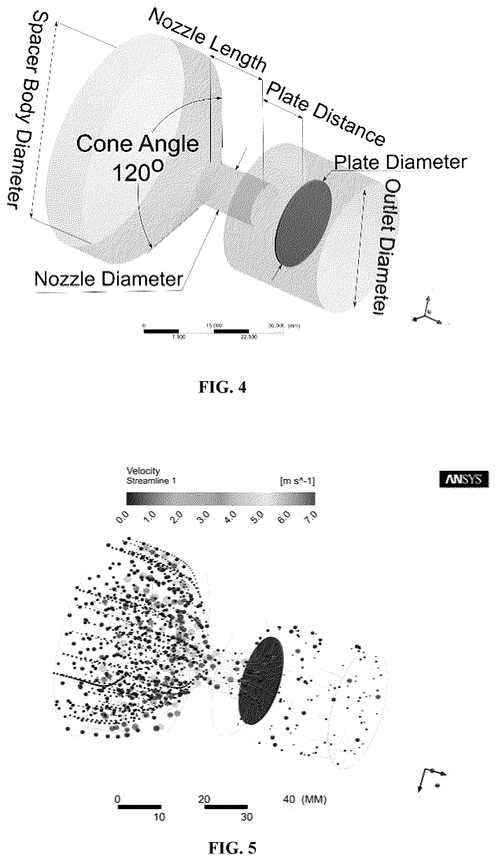

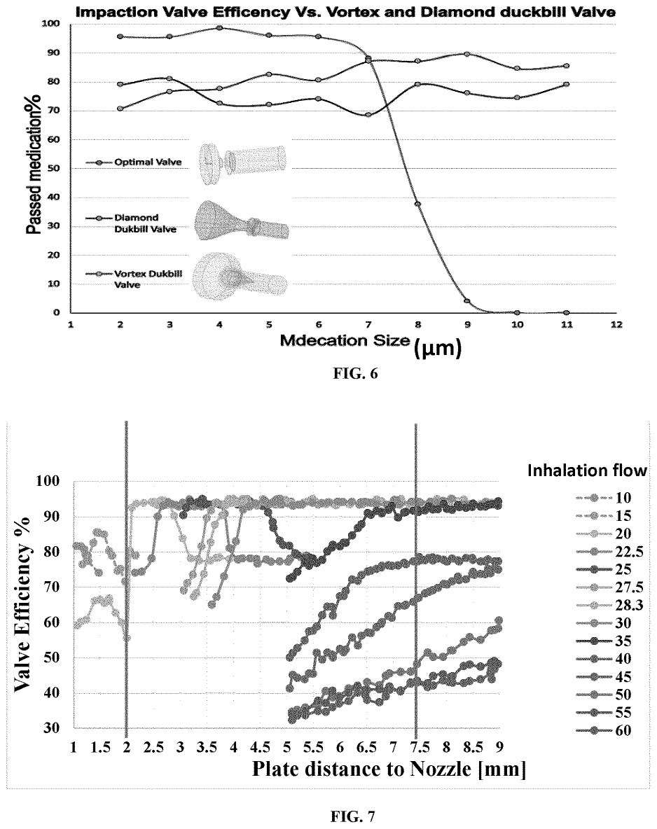

The 3D geometry model of the prototype with different valve parameters was also simulated in Ansys CFX. The turbulent model was set to Shear Stress Transport (SST) with the default parameters, and the Lagrangian method is used for particle modelling. To reduce the computational cost, the simulations only considered the end of the spacer body 14, including the cone end part, nozzle, and valve. FIG. 4 shows valve geometry of the simulation with portion of the spacer body with angled walls toward the valve inlet nozzle. The impact filter visualization for a simulated optimal valve is demonstrated in FIG. 5 with particle's diameters scaled 1000×. As shown in FIG. 5, large particles collide with the impact filter body and stick there, while small particles pass along with the flow streamlines. FIG. 6 shows simulated results of the impact valve efficiency to filter large particles comparing to known spacer's valves, such as Diamond and Vortex valves. The simulation results indicate superior performance of the impact valve of the present invention in filtering medications larger than 7 μm. As illustrated in FIG. 6, the valve with impact filter of the present invention filters more than 80% of the particles larger than 7 μm and sharply reaching to 0.0% after 9 μm, while the Diamond and Vortex valves filter around 23% and 13% of those particles respectively. Moreover, 96.2% of 6 μm particles and smaller pass the valve with impact filter, while the Diamond and Vortex valves only pass 75.7% and 77.5% of them.

As mentioned herein above, breathing flow rate is time-dependent and varies for different people with distinct conditions. A simulation and experimental study have been conducted to understand effect of the distance of the impact filter 18 to the inlet nozzle 12 on efficient drug delivery when the inhalation mass flow rate is changing in different patients, from infants and elderly ones to those with strong inhalation capability. Simulated numerical study is performed in the Ansys Workbench using the steady-state approach for drug delivery simulation in the human respiratory system. The turbulent model is set to Shear Stress Transport (SST) with the Ansys Workbench default parameters. The particles Lagrangian track method is used for particle modeling with one-way coupling, neglecting the effect of particles on the flow. FIG. 7 illustrates the valve's efficiency at various mass flow rates and different filter plate distance. The graph of FIG. 7 indicates the valve efficiency at different filter distance and various distinct mass flow rates. For example, at an inhalation mass flow rate of 15 L/min, the performance is 70% and it is achieved when the filter plate distance is approximately 1 mm. However, the best performance for this flow rate is 87% for 1.5 mm filter plate distance. The best performance of the valve is around 95% for the inhalation rates between 20 and 35 L/min, however, when the flow rate exceeds 40 L/min, the performance constantly drops and in 50 L/min it doesn't perform better than 60%. The simulation results demonstrate about linear relationship between inhalation mass flow rate and filter plate distance to inlet nozzle, as illustrated in FIG. 8 for flow rates between 10 to 35 L/min. The performance for inhalation mass flow rate of 40 L/min and above significantly drops as clearly illustrated in FIG. 7.

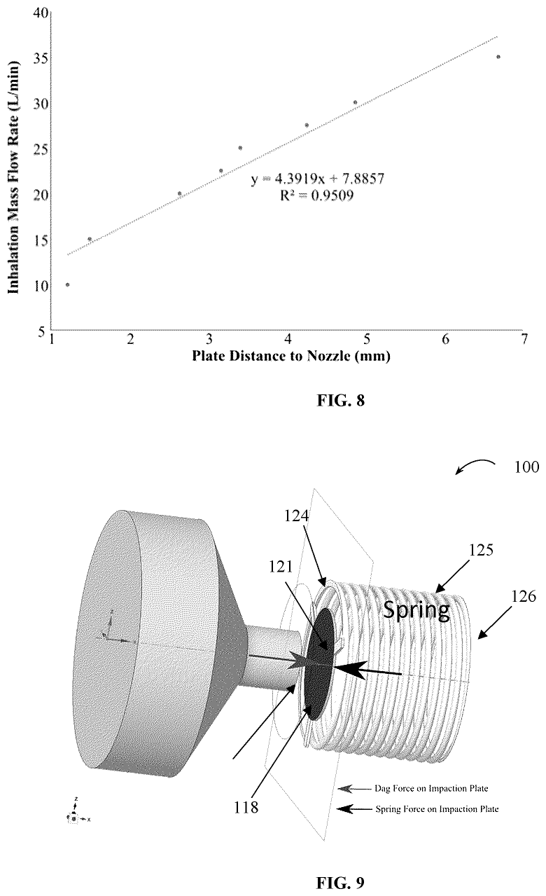

FIG. 9 is a schematic partial view of a valve 100 according to an embodiment of the present invention that is adjustable for different inhalation flow rates. Similarly, to valve 10 of FIG. 2, the valve 100 comprises an inlet nozzle 122, outlet 20 (not shown), a valve body 16 (not shown) and an impact filter 118 positioned in the valve body. The impact filter 118 comprises an impact plate 121 that is mounted to a spring 125 which is inserted in the inner cavity of the valve body. The spring 125 has a first end 124 facing the inlet nozzle 122 and a second end 126 facing the outlet 20. The impact filter 118 can be mounted to the first end 124 of the spring 125. The spring 125 can have a linear spring design that can be used to automatically adjust the filter plate 121 distance based on the flow rate.

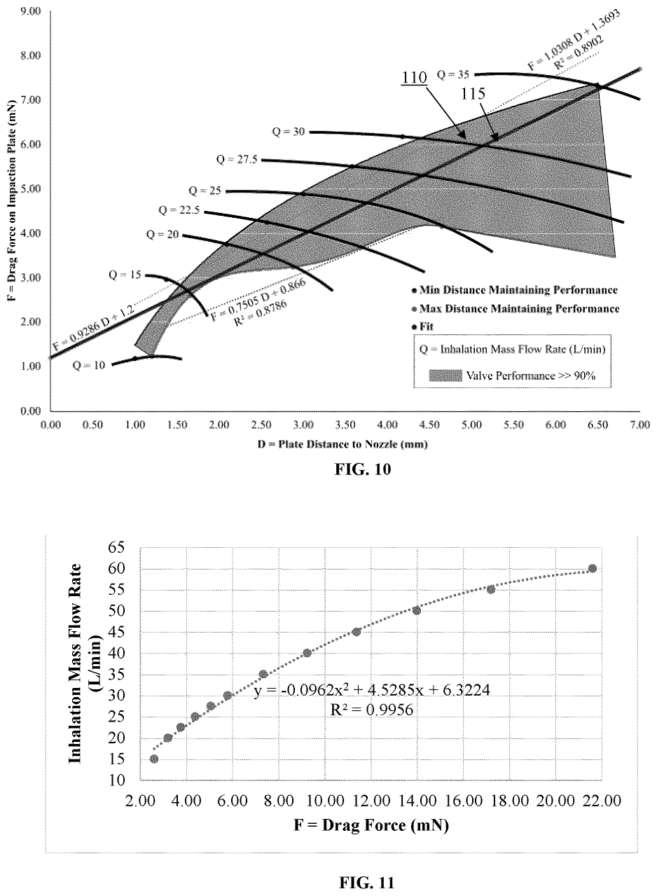

To determine the characteristics of the spring, an analysis has been conducted to find the relationship between the mass flow rate, filter plate distance to inlet nozzle, and the drag force applied to the filter plate by the airflow during inhalation. FIG. 10 illustrates a drag force on the filter impact plate at different filter plate distances to inlet nozzle for a range of the inhalation mass flow rate. If the filter impact plate 121 is positioned inside a green zone 110 for each flow rate, the valve 100 performance can be more than 90%. Blue line 115 represents the performance of the linear spring 125 with spring stiffness of 0.9286 mN/mm which is preloaded by 1.2 mN (spring compression of 1.3 mm). The impact plate 121 of the filter 118 is attached to the first end 124 of the spring 125 to regulate/adjust its movement based on the inhalation drag forces. The valve 100 can start to open when the drag force on the impact plate 121 overcomes the spring's force of 1.2 mN which is implemented by 1.3 mm pre-compressing of the spring 125 pushing the impact plate 121 away from the inlet nozzle 122 increasing the distance between the inlet 122 and the impact filter 118. As illustrated in FIG. 11, an inhalation mass flow rate can be proportional (or almost proportional) to the drag force that is equal to the spring force.

In some implementation, a spacer device shape and size can be optimized to reduce the amount of large particles at the outlet and therefore reduce deposition of such large particles in the oral cavity. The optimized shape and size of the spacer can trap large drug particles in the spacer itself and can allow more fine particles to the outlet for inhalation and penetration into deeper parts of the lungs. The spacer in general reduces the inhalers' injections speed and provides more fine particles to the user. When the pressurized medication suspension is injected from pMDI into the spacer, flow stream of aerosol particles is formed in the spacer's body. If the external and internal forces on the suspension droplet overcome its surface tension, breakup of the droplets occurs resulting in fine particles formation. This is affected by propellent, suspended medication and inhaler injection properties, such as inhaler nozzle dimensions and injection speed which can be controlled by controlling the size and shape of the spacer body.

Ansys software and fluid dynamics theory were used to study optimization of spacer's geometry and its impact on its performance. The research findings indicate that by optimizing a shape and size of the spacer's body, inhalation therapy can be significantly improved, potentially increasing the effectiveness of pMDIs for patients suffering from these lung diseases. The direct optimization method provided by ANSYS is used to conduct multi-objective optimization and a total of 1050 design points were tested. This method is chosen as it allows multiple objectives optimization, which is necessary for the optimization of the spacer's geometry. The optimal spacer body dimensions for two designs are presented in Table 2.

TABLE 2
Optimal spacer body results

| Variables[mm] | Design 1 | Design 2 |
|---|---|---|
| Body Length | 60.8 | 60.5 |
| Body Diameter | 33.5 | 22.6 |
| Cone Length | 13.1 | 1.9 |
| Mass Flow % | 60.1 | 57.3 |
| Ave. Particle Diameter Reduction % | 26 | 75 |

The results are spacer designs that would provide best performance in terms of drug delivery and particle transport and deposition. The optimal spacer body dimensions include variables such as body length, body diameter, cone length, mass flow, and average particle diameter reduction. These values represent the most efficient design of the spacer that can be used in inhalation therapy.

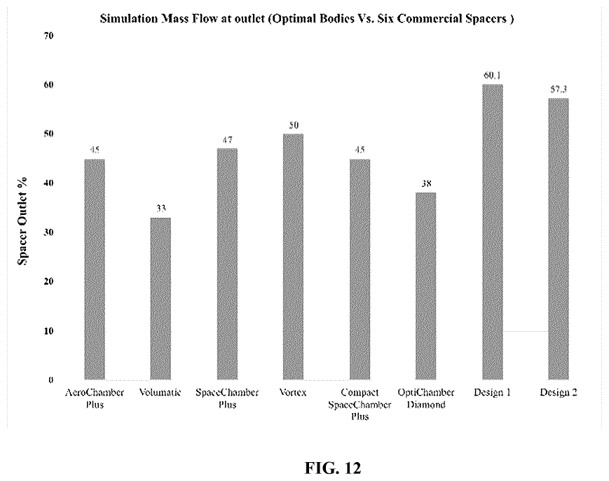

FIG. 12 illustrates the results of the two optimal spacer designs (Design 1 and Design 2) in comparison to six commercial spacer designs used in this study. The results depict that Design 1 has an outlet performance that is 10% higher than that of the Vortex spacer. This comparison highlights the improved performance of the optimized spacer designs, in terms of drug delivery, when compared to the commercial spacer designs. In addition, the tests show that Design 1 of the spacer has a particle size reduction of 26% at the outlet. The results also indicate that the Volumatic and Optichamber Diamond spacers perform better in terms of particle size reduction. Design 2 exhibits a particle size reduction of 75%, which is a significant improvement when compared to the other spacers in the study. Additionally, Design 2 has a good performance in drug delivery, with a rate of 57%. Overall, Design 2 shows a 7% better performance in terms of drug delivery, while also exhibiting excellent particle size reduction of 75%. The research findings indicate that by selecting a proper cylindrical shape and size of the spacer's body, inhalation therapy can be significantly improved, potentially increasing the effectiveness of pMDIs for patients suffering from these lung diseases.

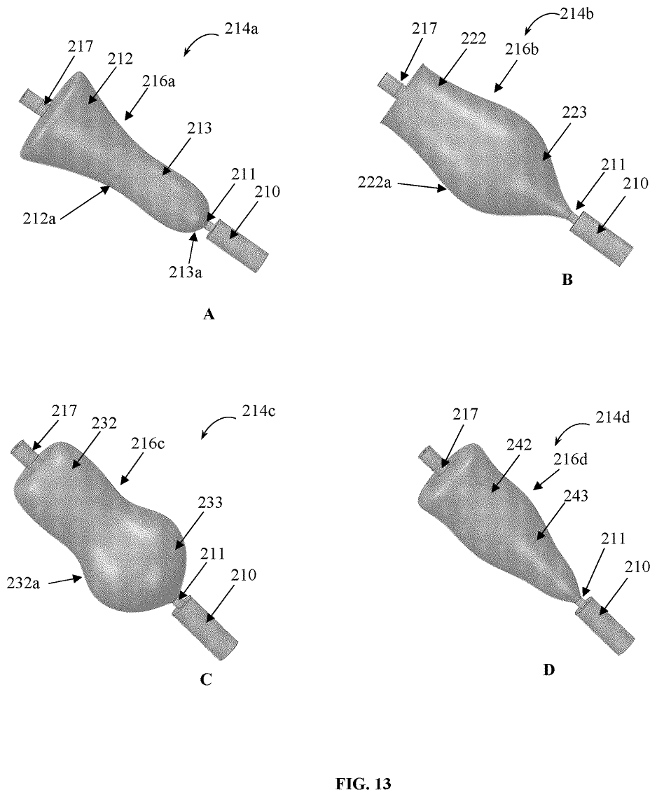

FIG. 13 illustrates four different optimized shapes of spacer body. The optimized spacer body designs characterize by modified cylindrical shape and have been shown to result in a significant improvement in the delivery of fine medication to the body outlets. Our studies have demonstrated that optimized design results in a 10% or greater improvement in comparison to traditional spacer bodies currently available on the market. This improvement has been achieved through the application of advanced mathematical concepts, including spline and bezier curve optimization, to the design of the spacer body. This novel approach to spacer body optimization represents a significant advancement in the field of inhalation therapy and has the potential to greatly improve the delivery and efficacy of inhaled medications. FIG. 13A illustrates a spacer 214a having a bell shape hollow body 216a with a spacer inlet 217 configured to be mounted to the nozzle of the pMDI canister and a spacer outlet 211 coupled to an impact valve 210. The impact valve can be similar to the impact valve 10 or 100 described herein above. The spacer body 216a can have a first part 212 with a funnel-like shape and a larger diameter in proximity to the spacer inlet 217 that decreases linearly to a distal end 212a of the funnel-like part 212. A second part 213 of the bell-shaped spacer body 216a is cylindrical with angled part 213a in proximity to the spacer outlet 211. FIG. 13B illustrates a spacer 214b having a cup-shaped hollow body 216b. The cup-shaped body 216b has a first part 222 coupled to the inlet 217 that is cylindrical which slightly flare out to a bigger diameter at the distal end 222a and a second part 223 that is funnel-like with diameter narrowing toward the outlet 211. FIG. 13C illustrates a spacer 214c having a pear-shaped hollow body 216c. The pear-shaped body 216c has a first part 232 coupled to the inlet 217 that is cylindrical which flare out to a bigger diameter at the distal end 232a and a second part 233 that is funnel-like with diameter narrowing toward the outlet 211. FIG. 13D illustrates a conically shaped spacer 214d having a hollow body 216d with a first part 242 coupled to the inlet 217 that is cylindrical and a second part 233 that is conical with diameter narrowing toward the outlet 211.

In some implementations, the valve 10, 100 can be designed and optimized for an inhalation mass flow rate of 28.3 L/min, which is a standard mass flow rate in the pharmaceutical industry. The conventional inhalation mass flow rate can be approximately equivalent to breathing 500 mL (an average tidal volume) in around 1s steadily. The valve 10, 100 can operate effectively between 15 and 35 L/min of inhalation mass flow rate and the position of the impact filter and the position of the impact filter can be adjusted between approximately 1.5 to 6.5 mm, depending to the inhalation mass flow rate, regulated, and adjusted by the spring 125.

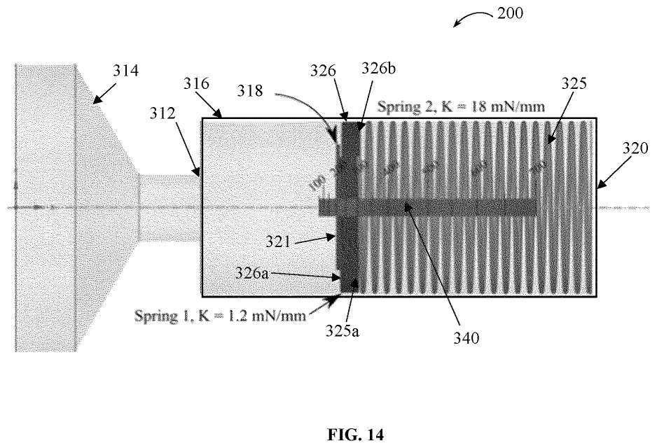

FIG. 14 illustrates an example of an impact valve 200 with an impact filter 318 that can be used as a valve holding chamber (VHC) in inhalers as medication filter to prevent large particles reaching user respiration parts during inhalation as well as a peak flow meter to evaluate breathing patterns of users in order to diagnose illnesses such as asthma, pulmonary fibrosis, cystic fibrosis, and chronic obstructive pulmonary disease (COPD). Thus, the impact valve 200 can be used as two-in-one device enhancing its versatility and clinical utility. The impact valve 200, similarly, to valves 10, 100, comprises an elongated body 316 with an inlet nozzle 312 and an outlet 320. The inlet nozzle 312 can be coupled to a spacer 314 and the outlet 320 can be designed as an mouthpiece. An impact filter 318 having an impact body 321 is mounted to a first spring 326 on its first end 326a facing the valve nozzle 312. The first spring 325 is coupled to a second spring 325 such that a second end 326b of the first spring is adjacent the engaged with a first end 325a of the second spring 325. Stiffness of the first spring 326 is lower than the stiffness of the second spring 325. The stiffness of the second spring 325 can be 10-20 times greater than the stiffness of the first spring 326. For example, stiffness of the first spring 326 can be about 0.9 mN/mm-1.2 mN/mm while stiffness of the second spring 325 can be about 15 mN/mm-18 mN/mm.

When the impact valve 200 is not in use both springs 325 and 326 are in relaxed state and the impact body is in close proximity to the valve nozzle. When the valve 200 is used as valve holding chamber (VHC) in inhalers, as the user inhales the air flow of the inhaled particles push on the impact body 321 of the impact filter 318 compressing the first spring 326 and moving the impact filter away from the nozzle 312, as illustrated in FIG. 14. Since the stiffness of first spring 326 is much lower than the stiffness of the second spring 325, the valve impact plate 321 will experience minimal drag force insufficient to engage or compress the second spring 325. Thus, and depending on the inhalation rate, the first spring 326 can be fully compressed while the second spring can experience barely any or small compression. The user can regulate his inhalation rate to keep the distance of the impact plate 321 to the inlet nozzle 312 less than 6.5 mm and more than 1.5 mm. Light or sound alert can be provided to alert the user when the inhalation mass flow rate is more than 35 L/mm. For example, a whistle (not shown) can be mounted to the valve 200 or the spacer 314 that can provide sound to alert the user to prevent them from speeding up the inhalation and keep the inhalation mass flow rate less than 35 L/min to maintain the valve optimality. When the valve 10 is a peak flow meter mode, it functions as a spirometry assessment device to determine lung function by measuring the breathing air velocity. As the air flow mass during exhalation breath puffs enter the valve through the mouthpiece at the outlet 320, it compresses the first spring 326 to a grater compression rate while the second spring, due to its greater stiffness, is insignificantly compressed and engaged to compensate for the force of exhalation peak flow, thereby facilitating accurate measurement of the flow rate by measuring the distance of the impact filter 318 from the valve nozzle. The valve 200 can further comprise a scale 340 that can be calibrated to quantify human expiratory peak flow. In addition, the valve system 200 is also capable of measuring inspiratory peak flow, enhancing its versatility and clinical utility.

While particular elements, embodiments and applications of the present disclosure have been shown and described, it will be understood, that the scope of the disclosure is not limited thereto, since modifications can be made by those skilled in the art without departing from the scope of the present disclosure, particularly in light of the foregoing teachings. Thus, for example, in any method or process disclosed herein, the acts or operations making up the method/process may be performed in any suitable sequence and are not necessarily limited to any particular disclosed sequence. Elements and components can be configured or arranged differently, combined, and/or eliminated in various embodiments. The various features and processes described above may be used independently of one another, or may be combined in various ways. All possible combinations and subcombinations are intended to fall within the scope of this disclosure. Reference throughout this disclosure to “some embodiments,” “an embodiment,” or the like, means that a particular feature, structure, step, process, or characteristic described in connection with the embodiment is included in at least one embodiment. Thus, appearances of the phrases “in some embodiments,” “in an embodiment,” or the like, throughout this disclosure are not necessarily all referring to the same embodiment and may refer to one or more of the same or different embodiments. Indeed, the novel methods and systems described herein may be embodied in a variety of other forms; furthermore, various omissions, additions, substitutions, equivalents, rearrangements, and changes in the form of the embodiments described herein may be made without departing from the spirit of the inventions described herein.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without operator input or prompting, whether these features, elements and/or steps are included or are to be performed in any particular embodiment. No single feature or group of features is required for or indispensable to any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The example calculations, simulations, results, graphs, values, and parameters of the embodiments described herein are intended to illustrate and not to limit the disclosed embodiments. Other embodiments can be configured and/or operated differently than the illustrative examples described herein.

1. An impact valve for a spacer, the valve comprising:
an inlet nozzle mounted to an outlet of a spacer body;
an outlet configured as a mouthpiece to be positioned into user's mouth;
an elongated tubular body having a wall defining an inner cavity extending between the inlet and the outlet; and
an impact filter positioned in the inner cavity of the valve body and having a diameter larger than a diameter of the inlet nozzle and smaller than a diameter of the outlet, the impact filter is secured in the inner cavity at a predetermined distance from the inlet nozzle,
wherein during inhalation, a majority of medication large particles are filtered out of a medication flow stream by impacting the impact filter while medication small particles pass to the outlet.

2. The valve of claim 1, wherein the impact filter is positioned at a pre-determined distance from the inlet nozzle, the pre-determined distance between the inlet nozzle and the impact filter is between 1.5 mm to 6.5 mm.

3. The valve of claim 1, wherein the impact filter comprises an impact body and a fastener connected to an inner side of the wall of the valve body and configured to secure the impact filter in the valve body, a gap/space is created between a peripheral edge of the impact body and the inner side of the wall through which the medication flow stream of particles passes to the outlet.

4. The valve of claim 3, wherein the fastener comprises a ring body connected to the inner side of the wall and at least one link connected to the ring body at one end and to the peripheral edge of the impact body at the opposite end bridging the gap/space.

5. The valve of claim 1, wherein the body of the valve comprises a number of nested pieces that slide one in relation to another to adjust a length of the body and therefore distance of the impact body to the valve inlet.

6. The valve of claim 5, wherein the body of the valve comprises a first part having a first end formed at the inlet nozzle and a second end, and a second part having a first end and a second end formed at the outlet, the second end of the first part being inserted into the second part through the first end of the second part, the impact filter being mounted in the second part of the body at a pre-determined distance between the first end and the second end of the second part, the first part is slidable between an extended position when the second end of the first part is in proximity to the first end of the second part and a nested position when an end face of the second end of the first part is adjacent to a portion of the impact filter, thereby adjusting the distance of the impact filter to the valve inlet nozzle.

7. The valve of claim 1, further comprising a first spring with a predefined spring stiffness mounted into the inner cavity of the valve body, the spring having a first end facing the inlet nozzle and a second end facing the outlet, the impact filter is mounted to the first end of the spring facing the inlet nozzle, wherein during inhalation a distance of the impact filter to the inlet nozzle is automatically adjusted based on user's inhalation flow rate pushing on the impact filter compressing the first spring and moving the impact filter away from the inlet nozzle.

8. The valve of claim 7, wherein the stiffness of the first spring is between 0.9 mN/mm-1.2 mN/mm.

9. The valve of claim 7, further comprising a second spring having a first end that engages the second end of the first spring, the second spring having a predefined stiffness that is greater than the stiffness of the first spring, wherein during inhalation the first spring is compressed due to lower stiffness moving the impact filter away from the inlet nozzle while the second spring is minimally compressed due to the greater stiffness therefore minimizing a drag force experienced in the valve.

10. The valve of claim 9, wherein the stiffness of the first spring is between 0.9 mN/mm-1.2 mN/mm and the stiffness of the second spring is between 15 mN/mm-18 mN/mm.

11. The valve of claim 1 incorporated in a pressurized meter dose inhaler.

12. The valve of claim 9 incorporated in a spirometry assessment device.

13. A pressurized meter dose inhaler comprising:
an inhaler container comprising a pressurized aerosolized medication and having an exit port to discharge the aerosolized medication;
a spacer having a first end coupled the exit port, a second end and a body extending between the spacer first end and its second end; and
a valve comprising:
an inlet nozzle mounted to the second end of the spacer;
an outlet configured as a mouthpiece to be positioned into user's mouth;
an elongated tubular body having a wall defining an inner cavity extending between the inlet and the outlet; and
an impact filter positioned in the inner cavity of the valve body and having a diameter larger than a diameter of the inlet nozzle and smaller than a diameter of the outlet,
wherein during inhalation, a majority of medication large particles are filtered out of a medication flow stream by impacting the impact filter while medication small particles pass to the outlet.

14. The pressurized meter dose inhaler of claim 13, wherein the spacer body is shaped and optimized to capture large particles, the spacer body having curved walls defining spacer inner cavity, the spacer body having bell-shaped body, cup-shaped body, pear-shaped body or conically shaped body.

15. The pressurized meter dose inhaler of claim 13, wherein the valve further comprises a first spring with a predefined spring stiffness mounted into the inner cavity of the valve body, the spring having a first end facing the inlet nozzle and a second end facing the outlet, the impact filter is mounted to the first end of the spring facing the inlet nozzle, wherein during inhalation a distance of the impact filter to the inlet nozzle is automatically adjusted based on user's inhalation flow rate pushing on the impact filter compressing the first spring and moving the impact filter away from the inlet nozzle.

16. The valve of claim 15, wherein the stiffness of the first spring is between 0.9 mN/mm-1.2 mN/mm.

17. The valve of claim 15, wherein the valve further comprises a second spring having a first end that engages the second end of the first spring, the second spring having a predefined stiffness that is greater than the stiffness of the first spring, wherein during inhalation the first spring is compressed due to lower stiffness moving the impact filter away from the inlet nozzle while the second spring is minimally compressed due to the greater stiffness therefore minimizing a drag force experienced in the valve.

18. The valve of claim 17, wherein the stiffness of the first spring is between 0.9 mN/mm-1.2 mN/mm and the stiffness of the second spring is between 15 mN/mm-18 mN/mm.

19. A peak flow meter comprising:
a valve comprising an inlet nozzle, an outlet configured as a mouthpiece to be positioned into user's mouth and an elongated tubular body having a wall defining an inner cavity extending between the inlet and the outlet;
a first spring with a predefined spring stiffness is mounted into the inner cavity of the valve body, the spring having a first end facing the inlet nozzle and a second end;
a second spring having a first end that engages the second end of the first spring and a second end facing the outlet, the second spring having a predefined stiffness that is greater than the stiffness of the first spring;
an impact body is mounted to the first end of the first spring facing the inlet nozzle and having a diameter larger than a diameter of the inlet nozzle and smaller than a diameter of the outlet; and
a scale coupled to an outer wall of the valve body and calibrated to measure air mass flow rate and quantify expiratory peak flow,
wherein an exhalation peak flow compresses the first spring to a greater compression rate due to its lower stiffness while the second spring is engaged and minimally compressed due to its greater stiffness compensating for a force of the exhalation peak flow, and wherein a peak flow rate is measured by measuring a distance of the impact body from the valve inlet nozzle.

20. The peak flow meter of claim 19, wherein the stiffness of the first spring is between 0.9 mN/mm-1.2 mN/mm and the stiffness of the second spring is between 15 mN/mm-18 mN/mm.
